(19) United States
(12) Patent Application Publication
(10) Pub. No.: US 2004/0267203 A1
(43) Pub. Date: Dec. 30, 2004

(54) Splittable cannula having radiopaque marker

(76) Inventors: Daniel J. Potter, Stillwater, MN (US); Richard E. Stehr, Stillwater, MN (US)

Correspondence Address: HEIMBECHER & ASSOCIATES, LLC., 390 UNION BLVD, SUITE 650, LAKEWOOD, CO, 80228-6512, US

(21) Appl. No.: 10/609,206

(22) Filed: Jun. 26, 2003

(51) Int. Cl. A61M 5/178; A61M 5/00; A61M 25/00

(52) U.S. Cl. 604/164.05; 604/264

(57) ABSTRACT

A splittable cannula including a radiopaque marker. Generally, the marker is placed at or near the distal end of the cannula. The radiopaque marker may be a unitary piece, or may consist of several portions. The sheath may include a sheath body having at least one sheath score line running along a portion of the sheath body, a radiopaque marker located at a distal end of the sheath body and abutting the sheath body, and at least one score line running along a portion of the radiopaque marker. The radiopaque marker may be located within the lumen of the sheath, or may be embedded in the sheath wall. Alternative embodiments may replace the marker score line with a notch or groove running along the length of the radiopaque marker. The notches, grooves, and/or score lines in both the sheath body and radiopaque marker serve to provide a weakened tear path.

### a. FIELD OF THE INVENTION

The invention relates generally to the field of medical instruments, and more particularly to a cannula having a radiopaque portion, for use during medical procedures.

### b. BACKGROUND ART

Several medical procedures require the introduction of one or more medical instruments into arteries or veins so that the medical instruments may be advanced to a body location requiring, for example, diagnosis or treatment. A sheath, or cannula, is often introduced into a patient's body to permit medical instruments (such as various types of catheters, leads, and so forth) to be inserted into the sheath and manipulated within the body. Accordingly, the cannula may also be referred to as an “introducer sheath.” The sheath effectively acts as a conduit, preventing damage to the wall of the vein or artery into which it is inserted. Similarly, the sheath may protect the region of the body to be diagnosed and/or treated. A hemostasis valve may be mounted to the end of the sheath located outside the patient's body in order to minimize blood flowing through the sheath and into the atmosphere.

In many medical procedures, the location of the sheath tip is important, insofar as the sheath must extend to the portion of the patient's body being diagnosed or treated. For example, ablation of a patient's heart requires precise placement of a catheter. Oftentimes, the catheter or sheath tip is tracked or located by electrophysiological guidance, fluoroscopy, or a combination of the two. In order to accurately track the catheter tip with fluoroscopy, a radiopaque material (i.e., a material reflecting or opaque to x-rays) is typically incorporated into the tip in some fashion. The radiopaque material generally takes the form of a coating or solid, metallic ring.

Many times, introducer sheaths or cannulas are also splittable. That is, they may be torn apart longitudinally while removed from a patient's body in order to more rapidly remove the sheath, and thus limit blood loss. However, the radiopaque material often inhibits splitting of the introducer sheath, especially when the radiopaque substance is in the form of a solid metal ring. Even coatings applied to a sh6ath may reduce the sheath's ability to split apart. Thus, many current introducer sheaths compromise between quick removal and detectability via x-rays during procedures.

Accordingly, there is a need for an improved introducer sheath having a radiopaque marker.

### SUMMARY OF INVENTION

Generally, one embodiment of the present invention takes the form of a splittable cannula or sheath, suitable for introduction into a blood vessel or other portion of a human body, including a radiopaque marker at its distal end and splittable along its length. The sheath may include a sheath body having at least one longitudinally extending sheath score line running along a substantial portion of the sheath body, a substantially cylindrical radiopaque marker located at a distal end of the sheath body and abutting the sheath body, and at least one longitudinally extending marker score line running along a substantial portion of the radiopaque marker. The radiopaque marker may be located within the lumen of the sheath, or may be embedded in the sheath wall. Similarly, alternative embodiments may replace the marker score line with a notch or groove running along the length of the radiopaque marker. The notches, grooves, and/or score lines in both the sheath body and radiopaque marker serve to provide a weakened tear path, thus facilitating rapid removal of the sheath and marker from a patient's body.

Generally, the sheath and marker score lines are substantially aligned, in order to permit relatively simultaneous splitting of the marker and the portion of the sheath surrounding the marker. Where a notch is used in lieu of a marker score line, the notch is typically similarly aligned with a sheath score line. Generally, many (although not necessarily all) embodiment of the cannula and radiopaque marker have two opposing score lines located diametrically opposite one another along the sheath and marker bodies.

In yet another embodiment, the radiopaque marker may be made of two or more discrete radiopaque portions, which may or may not be in direct contact with one another. For example, one embodiment of the radiopaque marker takes the form of two semicircular portions embedded within or affixed to the sheath wall at or near the distal tip. In such an embodiment, neither semicircular portion overlaps or contacts a sheath score line. Instead, the sheath score line or lines run along a gap between the semicircular portions. Accordingly, when the sheath is split, it tears between the radiopaque semicircular portions. This eliminated the need to actually break or tear through radiopaque material.

### SUMMARY OF DRAWINGS

FIG. 1 depicts a longitudinal cross-sectional view of a tip of a first embodiment of a splittable introducer sheath incorporating a radiopaque tube-shaped marker.

FIG. 2 depicts a cross-sectional view, taken along line 2-2 of FIG. 1, of the tip of the introducer sheath of FIG. 1.

FIG. 3 depicts a longitudinal cross-sectional view of a tip of a second embodiment of a splittable introducer sheath incorporating a radiopaque marker formed of discrete elements.

FIG. 4 depicts a cross-sectional view, taken along line 4-4 of FIG. 2, of the tip of the second embodiment of the introducer sheath, as shown in FIG. 3.

FIG. 5 depicts a longitudinal cross-sectional view of a tip of a third embodiment of a splittable introducer sheath incorporating a first embodiment of a radiopaque marker ring.

FIG. 6 depicts a cross-sectional view, taken along line 6-6 of FIG. 5, of the tip of the third embodiment of the introducer sheath, as shown in FIG. 5.

FIG. 7 depicts a longitudinal cross-sectional view of a tip of a splittable introducer sheath incorporating a second embodiment of a radiopaque marker ring.

FIG. 8 depicts a cross-sectional view, taken along line 8-8 of FIG. 7, of the tip of the introducer sheath, as shown in FIG. 7, depicting the second embodiment of the radiopaque marker ring.

FIG. 9 depicts a cross-sectional view of a tip of a splittable introducer sheath incorporating a third embodiment of a radiopaque marker ring.

FIG. 10 depicts a cross-sectional view of a tip of a splittable introducer sheath incorporating a fourth embodiment of a radiopaque marker ring.

FIG. 11 depicts a cross-sectional view of a tip of a splittable introducer sheath incorporating a fifth embodiment of a radiopaque marker ring.

FIG. 12 depicts a cross-sectional view of a tip of a splittable introducer sheath incorporating a sixth embodiment of a radiopaque marker ring.

### DETAILED DESCRIPTION OF PREFERRED EMBODIMENTS

Generally, one embodiment of the present invention comprises a sheath or cannula having one or more radiopaque marker materials placed along the sheath tip. The radiopaque material generally reflects x-rays, which in turn displays the location of the sheath tip on standard monitors in use in hospitals or other operating environments. Such monitoring of the tip location allows a doctor, surgeon, or other person to pinpoint the tip location, which in turn facilitates the general operation for which the sheath is used. As used herein, the terms “sheath” and “cannula” are interchangeable.

The radiopaque marker may be bonded to the inside of the sheath, along the lumen, or may be embedded within the sheath walls. Further, the radiopaque marker may be suspended within a polymer, or may be one or more solid, contiguous pieces of material. For example, the radiopaque marker may take the form of fine particles suspended in a polymer tube, or may be a ring of radiopaque substance bonded to the inner surface of the cannula. Exemplary radiopaque materials suitable for use with the present invention include metals such as platinum, tungsten, gold, or other metals opaque to x-rays, or polymeric materials designed to be x-ray opaque.

The cannula may be split along its longitudinal axis to facilitate removal from a patient's body. Such splitting is generally accomplished by scoring or weakening the sidewall of the cannula in order to provide a tear path along the cannula's longitudinal axis. The scoring be along the inside or outside walls of the sheath, and typically forms a score line.

When the radiopaque marker takes the form of a single contiguous piece, it may have a variety of cross-sectional shapes designed to facilitate splitting of the sheath. For example, a groove or notch may be provided and aligned with the cannula score lines, in order to reduce the amount of radiopaque material present along the weakened cannula walls. The notch may take a variety of forms, as discussed in greater detail below. Alternatively, the radiopaque marker may be serrated instead of grooved or notched. As with the grooves or notches, serrations may facilitate splitting of the sheath. Accordingly, the term “score line,” as used herein, is intended to embrace scores, notches, serrations, walls having reduced cross-sections, and so forth.

In addition to forming a contiguous element, mixture of particles, or suspension in a polymer, the radiopaque material may comprise multiple strips, pieces, or other elements arranged in such a manner as to provide opacity during fluoroscopy or other medical procedures. Such strips may be overlapping or placed sufficiently close to one another to provide the aforementioned opacity. In alternative embodiments, the radiopaque marker may be formed from a braided material.

Generally, no matter the exact form taken by the radiopaque material (whether particulate, multiple sections, or a single element), the material is sufficiently dense to be opaque when viewed from a variety of angles during fluoroscopy. Further, the radiopaque marker embodiments discussed herein generally extend from the distal end along a portion of the sheath lumen, and may extend along any distance of the lumen, up to and including the entirety of the lumen, in alternative embodiments.

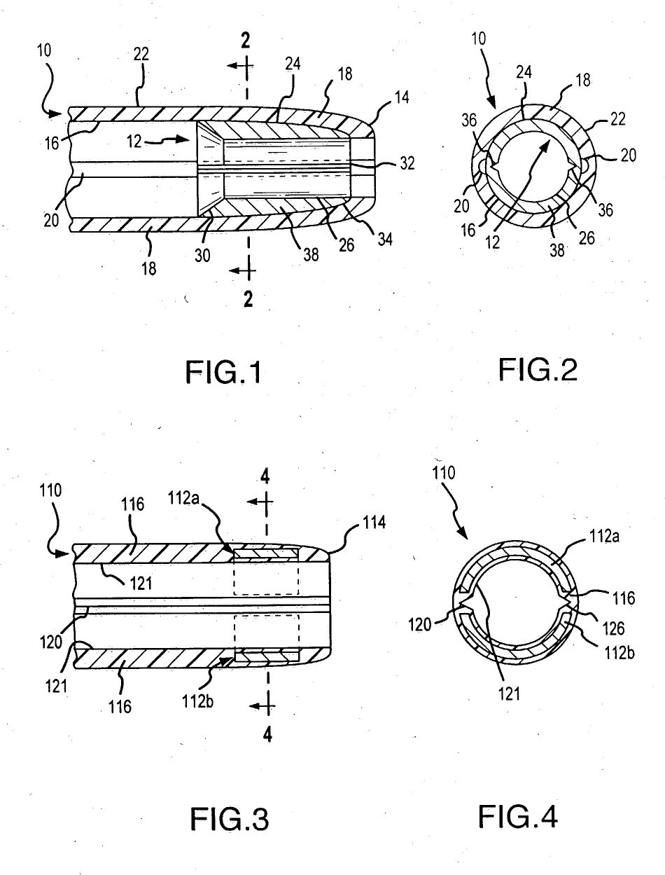

Turning now, to FIG. 1, a cross-section of a first embodiment of a splittable introducer sheath 10 incorporating a radiopaque marker 12 is depicted. Generally, the sheath is made of a polymer or other material suitable for introduction into a vein, artery, or other portion of a patient's body. The sheath is weakened along its longitudinal axis by at least one score line 20. Generally, such score lines extend substantially from a neck, or other point of attachment to a hemostasis valve (the “proximal end” of the sheath), to the opposite end of the introducer sheath 14 (the “tip” or “distal end” of the sheath). In the cross-section of the embodiment shown in FIG. 1, two score lines run along the sheath interior 16, each approximately opposite one another along the interior wall of the sheath, although only one is shown. In alternative embodiments, more score lines or grooves may be present, or the score lines may run along the exterior 22 of the sheath.

Typically, the entire length of the cannula 10 is splittable. This permits the cannula to be divided into multiple, separate portions as it is removed from the patient's body. Although specific reference is made herein to score lines 20, grooves, and/or weakened portions of the sheath sidewall, any structure permitting the division of the introducer sheath into two or more separate longitudinal pieces is contemplated as being usable with the present invention.

Extending along the interior of the distal portion of the cannula 10 is an interior tubular element 12 including radiopaque material. This tubular element (the “radiopaque tube”) is opaque to x-rays. Typically, the outer diameter of the radiopaque tube is approximately equal to the inner diameter of the cannula. Thus, when the radiopaque tube 12 is placed within the cannula 10, the outer wall 24 of the radiopaque tube contacts substantially all of the inner surface 26 of the cannula.

Cannula Having an Radiopaque Marker Attached to the Inner Cannula Wall

As may also be seen in FIG. 1, the inner wall 26 of the radiopaque tube 12 may taper along its proximal end, forming a funnel shape. The angular, annular wall 30 forming this tapering portion may assist in guiding leads, instruments, or other items inserted into the introducer sheath to the distal tip 14 of the sheath 10. The radiopaque tube 12 may extend along any portion of the sheath 10 desired, so long as the length and thickness of the radiopaque tube is sufficient to be detected during typical fluoroscopy operations when viewed at various angles of exposure. The radiopaque tube (also referred to as a “radiopaque marker”) is generally defined by a body having a substantially cylindrical sidewall with inner 26 and outer 24 surfaces.

Similarly, the distal end 32 of the radiopaque tube 12 may have a curved or shaped outer wall 34, the curve or shape generally corresponding to the form of the lumen 16 or inner wall of the cannula 10. By matching the shapes of the walls, a greater bonding surface between the radiopaque tube 12 and cannula may be achieved.

FIG. 2 depicts a lateral cross-section of the splittable introducer sheath 10 shown in FIG. 1. The cross-section shown in FIG. 2 is taken along line 2-2 of FIG. 1. Generally, as depicted in FIG. 2, the radiopaque tube/marker 12 and sheath 10 both include at least one score line 36, 20 or other area having a weakened or narrowed lateral cross-section.

Herein, the score lines running along the radiopaque tube 12 are referred to as “marker score lines 36” or “radiopaque tube score lines.” The score lines of the radiopaque tube and cannula 10 are typically aligned with one another, in order to facilitate splitting the radiopaque tube in the same direction and along the same plane as the cannula. That is, the marker score lines 36 are typically within the plane defined by the score lines 20 of the cannula itself. Further, the radiopaque tube score lines may extend only partially through the radiopaque tube sidewall 38, or may extend entirely therethrough, as shown in FIG. 2. Where the marker score lines 36 extend completely though the radiopaque tube, the score lines may abut the score lines 20 formed on the inner sidewall 16 of the introducer sheath. Where the score lines 36 extend only partially through the radiopaque tube, the score lines may be on the interior 26 or exterior 24 of the tube sidewall 38. FIG. 2 depicts the score lines along the interior of the radiopaque tube sidewall.

Since the radiopaque tube 12 is bonded to portion the interior wall 16 of the cannula 10, the radiopaque tube/marker and longitudinal section of the cannula enveloping the marker generally split substantially simultaneously.

Typically, regardless of whether the score lines 36 are along the interior 26 or exterior 24 of the radiopaque tube 12, the score lines defined in both the radiopaque tube and sheath 10 are hollow, rather than filled in with a polymer or binding agent. In alternative embodiments, a binding material, polymer, or other element having a weaker resistance to shear stress created by splitting the sheath along the score line(s) 20, 36, may at least partially fill in one or more of the score lines mentioned herein.

The radiopaque tube 12 may be made of a variety of radiopaque materials. For example, the tube 12 may be made of radiopaque particulate suspended or otherwise mixed in a polymerized substance, such as barium sulfate, an x-ray impenetrable metal, or any other radiopaque substance that may be bonded to the inner wall of the introducer sheath. As described in more detail below, in the section entitled “Method of Constructing a Cannula Having a Radiopaque Marker,” the bonding between the radiopaque tube and cannula 10 may be achieved through thermal, sonic, chemical, mechanical, or adhesive bonding, or any other bonding means known to those skilled in the art. The exact bonding means may vary, depending on the composition of the radiopaque tube 12 and sheath 10.

Cannula Having an Radiopaque Marker Embedded in the Cannula Wall

FIG. 3 depicts a longitudinal cross-section of a second embodiment of a splittable introducer sheath 110 having a radiopaque marker 112 placed within its distal end 114. In the embodiment shown in FIG. 3, the radiopaque marker comprises multiple, physically separate elements, instead of a unitary piece. Generally, the embodiment includes at least one piece of radiopaque material for each segment of the cannula wall 116, where a “segment” is defined as a portion of a sheath extending between two score lines 120. Thus, where a sheath has two score lines (as is the case with the embodiment of FIG. 3), the radiopaque marker 112 may consist of two discrete radiopaque elements 112a, 112b. Where, for example, the sheath is divided into three pieces by three unique score lines, three discrete radiopaque elements may be embedded in the sheath walls.

Generally, it should be noted that a sheath and corresponding radiopaque marker may have any number of score lines, as necessary, to facilitate splitting of the sheath and marker. For example, a sheath and marker may each have four score lines, each offset by ninety degrees, to split the sheath and marker into four pieces. Further, the sheath and marker may have differing numbers of score lines. For example, a sheath may have two score lines, while the radiopaque marker used with the sheath may have twenty or thirty score lines. In such an embodiment, the plurality of score lines along the marker may permit less precise alignment of any single score line with the sheath. If the radiopaque marker is scored every five millimeters along its circumference, for example, then the sheath score line will never be more than two and a half millimeters distant from the nearest marker score line. Such distance between the sheath and marker score lines is sufficiently minimal to permit breaking of both the sheath and the radiopaque marker simultaneously. Accordingly, a marker with this score pattern may be inserted within the sheath without reference to the sheath score lines.

In the present embodiment, the radiopaque material is typically embedded within the walls 116 of the sheath 110, rather than bonded to the interior sheath wall 116, as with the embodiment of FIG. 1. Any of the shapes and configurations discussed herein, however, may be bonded to the interior sheath wall 120 and are suitable for use with the embodiment of FIG. 1.

Still with reference to FIG. 3, a lateral cross-sectional view of the present embodiment of a distal tip 114 is depicted, showing the radiopaque marker portions 112a, 112b embedded in the sheath walls 116. The extension of each marker portion 112a, 112b, along the wall of the introducer sheath and terminating prior to the scored groove 120, is shown in phantom to aid in understanding the reach and extent of the marker's circumferential coverage. Typically, the portion of the sheath sidewall 116 along which the scored groove is formed does not contain any embedded radiopaque marker material. This lack of material along the scores facilitates tearing and removal of the sheath from a patient's body, without requiring the radiopaque marker itself to be torn.

FIG. 4 displays a lateral cross-section of the distal tip 114 of the introducer sheath 110 of FIG. 3, taken along line 4-4. FIG. 4 more clearly shows the radiopaque marker 112 material embedded in the sheath walls 116, along with the lack of marker material in the sheath sidewall at the portions of the sidewall having a scored line 120.

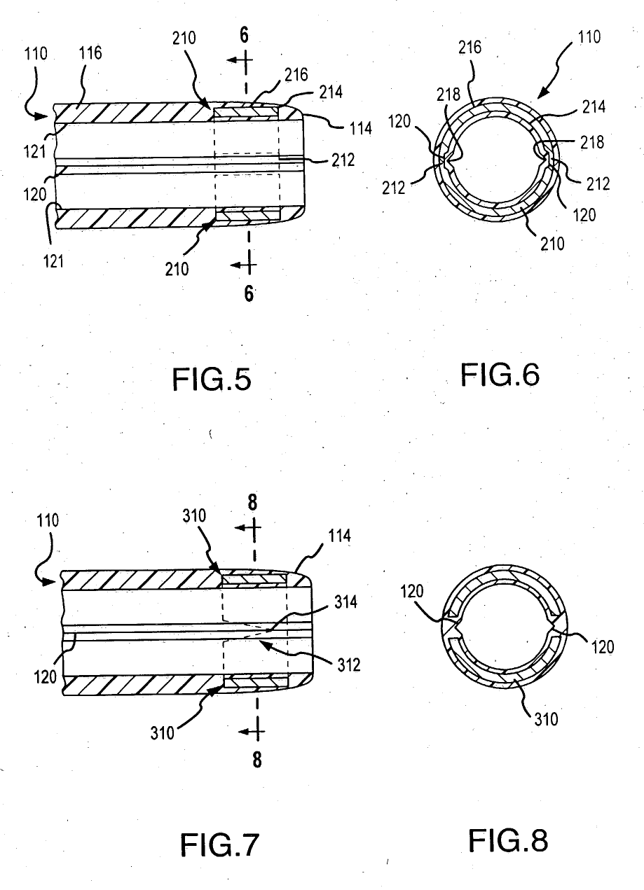

FIG. 5 displays a cannula 110 having a first embodiment of a radiopaque marker ring 210. In this embodiment, the marker is again embedded in the sidewall 116 of the sheath. Here, however, the radiopaque marker 210 is generally of unitary construction, and extends throughout the sidewall of the sheath, including running along the scored portion 120 of the sheath sidewall 116. The marker is generally ring-shaped.

In order to facilitate splitting the sheath 110, the present embodiment's radiopaque marker 210 may be grooved, scored, serrated, or provided with a sidewall having weakened or reduced material cross-sections 212 generally corresponding to the location of the score lines 120, grooves, or other splitting means running along the longitudinal axis of the sheath. These various options are collectively referred to as “marker score lines,” 212, and facilitate splitting the radiopaque marker. Generally, because the marker is embedded in the sidewall 116 of the sheath, the marker and portion of the sheath 110 surrounding the marker are split substantially simultaneously. The alignment of the sheath score lines 120 and marker score lines 212 reduce the force necessary to split the distal tip 114 of the sheath. As with previously-discussed embodiments, the marker score lines may be formed on either the interior 214 or exterior 216 of the marker sidewall. Generally, the depression or cavity 218 defined by the marker and/or cannula score lines remains hollow, although alternative embodiments may at least partially fill in one or more of these score lines with a binding material, polymer, or other element having a weaker resistance to shear stress created when splitting the sheath 110.

In alternative embodiments, a series of overlapping or closely spaced radiopaque markers may be used, instead of a single radiopaque marker ring 210. In such, an embodiment, the radiopaque markers lying in a plane defined by a line connecting the sheath score lines 120 may be scored or grooved 212 as described above. In other words, radiopaque markers embedded in the sheath sidewall 116 directly behind the score lines 118 may also be scored or have a reduced cross-section.

FIG. 6 depicts a lateral cross-section of the sheath 110 of FIG. 5, taken along line 6-6 of FIG. 5. The alignment of the scores 120, 212 on the sheath interior 116 and radiopaque marker 210 are shown. Although the radiopaque marker is shown as scored along its interior sidewall 214, the exterior sidewall 216 may alternatively be scored, or both sidewalls may be, scored.

FIG. 7 depicts a cross-sectional view of a tip of a splittable introducer sheath 110 incorporating another embodiment of a radiopaque marker ring 310. The radiopaque marker ring embedded in the sheath 110, as shown in FIG. 7, is not grooved or scored longitudinally. Instead, a V-shaped notch 312 is formed in the marker ring 310 along the ring sections corresponding to the sheath groove 120. Accordingly, the amount of marker material resisting splitting of the sheath along the score or groove is minimized. In turn, this permits, easier splitting of the cannula and radiopaque marker ring.

Many different notch shapes may be incorporated into the radiopaque annular marker ring 310, several of which are discussed below with respect to FIGS. 8-12. It should be noted that, for each of the marker rings discussed, the notch 312 generally underlies or is co-planarly located with at least one sheath score line 120. For each of these marker rings, the lack of radiopaque material in the formed notch 312 is insufficient to prevent overall opacity of the radiopaque marker during fluoroscopy or other exploratory procedures.

FIG. 8 depicts a lateral cross-sectional view of the cannula tip 114 of FIG. 7, taken along line 8-8 through the marker ring notch 312. The lack of radiopaque marker material underlying or adjacent to the sheath groove 120 is clearly shown at the cross-section. Were the cross-section taken closer to the distal end of the marker 310, the radiopaque material of the present embodiment would form a continuous ring. The notch in the marker ring 310 of FIGS. 7 and 8 generally terminates in a point 314.

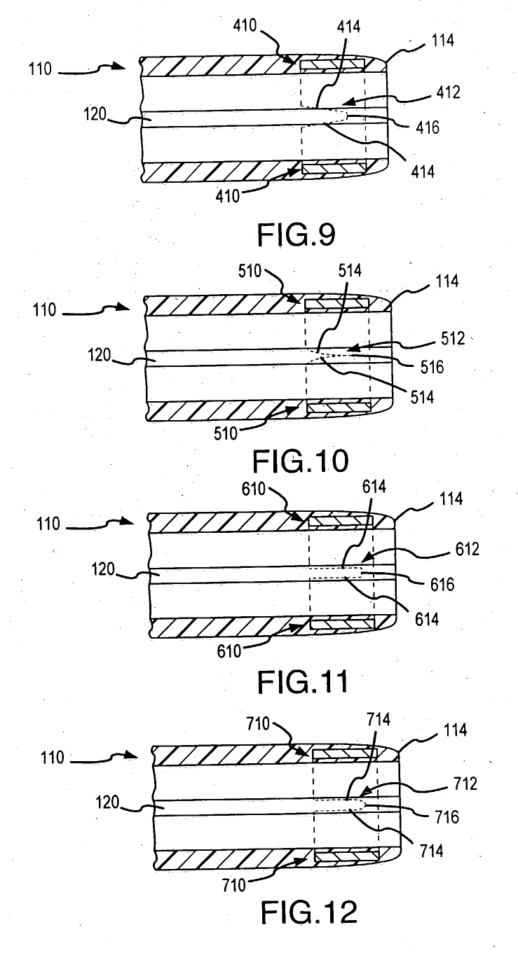

FIG. 9 depicts a longitudinal cross-sectional view of the distal portion 114 of a splittable introducer sheath 110 incorporating a third embodiment of a radiopaque marker ring 410. It should be noted that the marker embodiment discussed with respect to FIGS. 1 and 2 is a tube. Similarly, the marker embodiment shown in FIGS. 3 and 4 is made of separate pieces. Accordingly, the embodiments shown in FIGS. 5 and 6 is the first marker ring embodiment. FIGS. 7 and 8 depict the second ring embodiment, and FIG. 9 depicts the third ring embodiment. Here, the notch 412 has sloped walls 414. These walls terminate in a substantially flat surface 416 parallel to the distal end of the marker ring instead of coming to a point, as in the embodiment of FIGS. 7 and 8.

FIG. 10 depicts a longitudinal cross-sectional view of the distal portion 114 of a splittable introducer sheath 110 incorporating a fourth embodiment of a radiopaque marker ring 510. In this embodiment, the notch 512 has inwardly curved sidewalls 514 (i.e., sidewalls curving towards the center of the notch). The notch sidewalls meet at a point 516, but may terminate in a flat or curved surface in alternate embodiments.

FIG. 11 depicts a longitudinal cross-sectional view of the distal portion 114 of a splittable introducer sheath 110 incorporating a fifth embodiment of a radiopaque marker ring 610. The embodiment of FIG. 11 includes a marker ring 610 having a notch 612 with parallel sidewalls 614 and a base wall 616 perpendicular to the sidewalls. As with the embodiments discussed with respect to FIGS. 7-10, the lack of radiopaque material along the notch facilitates splitting of the sheath along the sheath score line 120.

FIG. 12 depicts a longitudinal cross-sectional view of the distal portion 114 of a splittable introducer sheath 110 incorporating a sixth embodiment of a radiopaque marker ring 710. Although this embodiment of the marker ring includes substantially parallel notch 712 sidewalls 714, like those shown in FIG. 11, the marker ring of FIG. 12 has a curved base wall 716 instead of a flat one 616.

It should be noted that the various embodiments of the cannula 10 and radiopaque marker 123 discussed herein, and depicted in the various figures, may have a variety of measurements, either standing alone or in relation to one another. For example, the thickness of the lumen sidewall 16 and/or radiopaque markers 12 may vary from embodiment to embodiment, as may the diameter of the lumen. Accordingly, the embodiments shown in the figures are for illustrative purposes only, and should not be construed as depicting exact measurements or relationships between the sizes of various elements of the present invention.

Method of Constructing A Cannula Having a Radiopaque Marker

The general procedure for constructing a splittable sheath 10 including a radiopaque marker 12 designed to facilitate sheath splitting will be given with reference to the embodiment shown in FIGS. 1 and 2, although it should be understood that this method of construction is generally applicable to all embodiments discussed herein. Initially, the splittable sheath 10 tubing is extruded and scored. Next, a marker 12 may be inserted into the distal end 14 of the sheath tubing, prior to forming the sheath tip. Once the ring is lodged within the tip, the sheath tip and ring may be inserted into a tip forming die.

In more detail, a die pin or rod may be run along the length of the sheath 10 from its proximal end to its distal end 14. Generally, the die pin is circular in cross-section, approximately equal in diameter to the inner sheath wall or lumen 16 diameter, and has opposing, outwardly-extending projections. As these projections run along the interior sheath sidewall 16, they score the sidewall, thus creating the score lines 20 previously referenced. As the die pin passes to the distal end 14 of the sheath 10, it also scores the radiopaque marker 12.

Where the sheath 10 and marker 12 score lines 20, 36 are not immediately adjacent (for example, as shown in FIG. 2A), both the sheath and radiopaque marker may be scored prior to inserting the marker into the sheath.

Once the scoring process is complete and the marker 12 is snugly nested in the sheath 10, the marker and sheath may be bonded. Exemplary bonding methods include thermal, sonic, and chemical bonding.

Finally, the sheath tip 14 is formed. The sheath tip 14 may be formed by injecting or extruding additional polymer material, deforming via heat or mechanical means a portion of the tube, overmolding additional polymer material in the shape of the tip 14, and so forth. If the tip is overmolded, the overmold material may be deposited around a portion of the tube, or alternatively the marker 12 may be only partially inserted into the tube and the overmold material placed around the marker.

Yet another manufacturing method is particularly suitable for creating the embodiments discussed with respect to FIGS. 3-12, and the method is discussed with reference to the embodiment of FIGS. 3-4. First, a splittable cannula 110 having a radiopaque marker 112a, 112b may be made by first extruding the sheath, as mentioned above. The extruded sheath 110 may be slit longitudinally, with the slit beginning at the sheath's distal end 114 and extending along a portion of the sheath sidewall 116. The slit is typically circular in lateral cross-section, and divides the distal end 114 of the sheath into an inner and outer ring.

The radiopaque marker 112 (or its constituent elements 112a, 112b) may then be inserted into the slit, effectively being placed between the inner and outer rings of polymer material at the distal tip 114 of the sheath 110. The inserted marker may be made of strips, braided material, a solid ring such as those discussed with respect to FIGS. 5-12, or separate pieces such as those discussed with respect to FIGS. 3 and 4. Generally, the marker 112 is placed inside the sheath 110 in such a manner that the marker scores, serrations, notches, reduced cross-sectional areas, and so forth align with the sheath score lines 120. In alternative embodiments employing a multi-serration or multi-score marker, the exact alignment of marker and sheath score lines may not be necessary. The marker 112 is typically inserted far enough into the sheath 110 that it is entirely encapsulated by the sheath, although alternative embodiments may vary this procedure.

Once the marker 112 is properly positioned, the inner and outer rings of the sheath 110 may be thermally or chemically bonded to one another to seal the marker inside the sheath. The tip 114 may then be formed as previously discussed, or the bonding process may simultaneously form the tip.

As will be recognized by those skilled in the art from the foregoing description of embodiments of the invention, numerous variations on the described embodiments may be made without departing from the spirit and scope of the invention. For example, the configuration of the radiopaque material may vary from embodiment to embodiment, as may its composition. Further, while the present invention has been described in the context of specific embodiments and processes, such descriptions are by way of example and not limitation. Accordingly, the proper scope of the present invention is specified by the following claims and not by the preceding examples.

What is claimed is:

1. A sheath having a distal end opaque to x-rays, comprising:
a sheath body, said sheath body comprising a first longitudinally extending sheath score line running substantially along a length of said sheath body;
a radiopaque marker located at a distal end of said sheath body, said radiopaque marker comprising a substantially cylindrical marker body; and
at least one longitudinally extending marker score line running substantially along a length of said radiopaque marker;
wherein said radiopaque marker abuts said sheath.

2. The sheath of claim 1, further comprising:
a second sheath score line running substantially along said length of said sheath body; and
a second marker score line running substantially along said length of said radiopaque marker.

3. The sheath of claim 2, wherein said first and second sheath score lines are located on an exterior surface of said sheath body.

4. The sheath of claim 2, wherein:
said first and second sheath score lines are located approximately opposite one another along an interior wall of said sheath body; and
said first and second marker score lines are located approximately opposite one another along said marker body.

5. The sheath of claim 4, wherein:
said first sheath score line and said first marker score line are aligned; and
said second sheath score line and said second marker score line are aligned.

6. The sheath of claim 5, wherein:
said radiopaque marker is located within said sheath body; and
said radiopaque marker is bonded to said sheath body.

7. The sheath of claim 6, wherein said radiopaque marker is thermally bonded to said sheath body.

8. The sheath of claim 5, wherein said radiopaque marker is embedded in said sheath body.

9. A sheath having a distal end opaque to x-rays, comprising:
a sheath body, said sheath body comprising a first longitudinally extending sheath score line running substantially along a length of said sheath body;
a radiopaque marker located at a distal end of said sheath body; said radiopaque marker comprising a substantially cylindrical marker body; and
at least one longitudinally extending marker notch running substantially along a length of said radiopaque marker;
wherein said radiopaque marker abuts said sheath.

10. The sheath of claim 9, further comprising:
a second sheath score line running substantially along said length of said sheath body; and
a second marker notch running substantially along said length of said radiopaque marker;
said first and second sheath score lines are located approximately opposite one another along an interior wall of said sheath body; and
said first and second marker notches are located approximately opposite one another along said marker body.

11. The sheath of claim 10, wherein:
said first sheath score line and said first marker notch are aligned; and
said second sheath score line and said second marker notch are aligned.

12. The sheath of claim 11, wherein said radiopaque marker is embedded within said sheath body.

13. The sheath of claim 12, wherein said first marker notch is V-shaped.

14. The sheath of claim 12, wherein said first marker notch is U-shaped.

15. The sheath of claim 12, wherein said first marker notch comprises:
first and second sidewalls, said first and second sidewalls substantially parallel to one another; and
a base wall joining said first and second side walls, said base wall substantially perpendicular to said first and second side walls.

16. The sheath of claim 12 wherein said first marker notch comprises a first and second curved sidewall, said first and second curved sidewalls meeting at a common point.

17. A sheath having a distal end opaque to x-rays, comprising:
a sheath body, said sheath body comprising at least one longitudinally extending sheath score line running substantially along a length of said sheath body;
a radiopaque marker located at a distal end of said sheath body, said radiopaque marker comprising a first marker half, said radiopaque marker defining a gap substantially aligned with said sheath score line; and
wherein said radiopaque marker abuts said sheath.

18. The sheath of claim 17, wherein said gap runs along an entirety of said first marker half.

19. The sheath of claim 17, wherein said radiopaque marker body further comprises:
a second body half; and
a second gap, said first and second gaps defining said first and second body halves; wherein
said first and second body halves are completely embedded in said sheath body.

20. The sheath of claim 19, wherein said radiopaque marker is made from a material from the group consisting of a particulate-laden polymer, barium sulfate, gold, platinum, and tungsten.
